(19) United States
(12) Patent Application Publication
(10) Pub. No.: US 2007/0232893 A1
(43) Pub. Date: Oct. 4, 2007

(54) Probe, image diagnostic system and catheter

(75) Inventor: Hiromichi Tanioka, Kanagawa (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Tokyo (JP)

(21) Appl. No.: 11/730,302

(22) Filed: Mar. 30, 2007

(30) Foreign Application Priority Data
Mar. 31, 2006 (JP) 2006-099927

(51) Int. Cl. A61B 5/05; A61B 8/00; A61B 6/00

(52) U.S. Cl. 600/407; 600/437; 600/476

(57) ABSTRACT

A probe which is adapted to repeatedly transmit and receive signals during radial scanning within a body cavity to acquire reflected signals and transmit the reflected signals to an image diagnostic apparatus which, on a basis of the reflected signals, forms and outputs a tomographic image of the body cavity and biotissue surrounding the body cavity includes a hollow shaft for transmitting rotational drive force to perform the radial scanning, and a transmission line extending along the shaft to transmit the reflected signals to the image diagnostic apparatus. The shaft receives the rotational drive force via a torque limiter which possesses a thickness which is non-uniform in a circumferential direction at a part of the torque limiter along a length of the torque limiter.

### TECHNICAL FIELD

This invention generally relates to a probe, an image diagnostic system and a catheter.

### BACKGROUND OF THE INVENTION

Image diagnostic systems have been used for diagnosing arteriosclerosis, for preoperative diagnosis upon coronary intervention by a high-performance catheter such as a dilatation catheter (i.e., balloon catheter) or stent, and for assessing postoperative results.

Examples of these image diagnostic systems include intravascular ultrasound (IVUS) imaging systems. In general, the intravascular ultrasound imaging system is constructed to control an ultrasonic transducer to perform radial scanning within a blood vessel, to receive a reflected wave(s) (ultrasound echoes) reflected by biotissue (e.g., the blood vessel wall) by the same ultrasonic transducer, to subject the reflected waves to processing such as amplification and detection, and then to construct and display a tomographic image of the blood vessel on the basis of the intensities of the received ultrasound echoes.

In addition to these intravascular ultrasound imaging systems, optical coherence tomography (OCT) imaging systems have been developed in recent years for use as image diagnostic systems. In an OCT imaging system, a catheter with an optical fiber incorporated therein is inserted into a blood vessel. The distal end of the optical fiber is provided with an optical lens and an optical mirror. Light is emitted in the blood vessel while radially scanning the optical mirror arranged on the side of the distal end of the optical fiber, and based on light reflected from biotissue forming the blood vessel, a tomographic image of the blood vessel is then constructed and displayed.

In addition, improved OCT imaging systems have been proposed in recent years which make use of a wavelength swept light source.

Thus, a number of systems which differ in detection principle have been finding utility as image diagnostic systems. These systems are all characterized in that a signal transmitting and receiving portion is positioned on a distal end of a driveshaft through which rotational drive force is transmitted, the signals are transmitted and received through a transmission line (transmission line for electrical signals or optical signals) in the driveshaft, and radial scanning is performed to extract tomographic images.

Such a driveshaft presents a potential problem in that an overload may be applied to the distal end portion of the driveshaft if a catheter (i.e., probe) is trapped at a constricted point or an exterior sheath covering the driveshaft is damaged when the catheter is inserted into a body cavity such as a blood vessel or lumen.

To address this, JP-A-H7-184888 proposes a construction in which a rotation control system operating a driveshaft is provided with a current limiting circuit which cuts off current to a motor when a detected current value has exceeded a preset current value and a measured drive time has become longer than a preset standard time. The rotation control system is, therefore, constructed such that the rotation of the motor stops only when an overload has applied to the driveshaft.

According to JP-A-H10-66696, on the other hand, a coupler is connected to a rotary shaft of a rotational drive source, a plug is connected to the side of a driveshaft and carries a permanent magnet embedded therein, and a rotatable contact of ferromagnetic material is rotatably secured on the coupler. These coupler, plug and rotatable contact are constructed such that when a load torque is not greater than a predetermined value, the coupler and the plug are coupled together via the contact under the attractive force of the permanent magnet. On the other hand, when a load torque is greater than the predetermined value, the contact is allowed to slide on the outer circumferential surface of the plug and the coupling between contact and the plug is cancelled to move the contact under centrifugal force.

To date, a variety of proposals have been made to avoid the application of an overload to a distal end portion of a driveshaft as described above, because it is generally common to consider that, when a device for rotating the driveshaft is used in a blood vessel or a lumen, the application of an overload to the distal end of the driveshaft suggests the possible occurrence of a certain trouble. It is, therefore, desired for a rotation control system or mechanism to have such a construction that upon application of an overload, the rotation of the driveshaft can be instantaneously stopped to minimize damages to a biotissue.

The system mentioned above which employs a rotation limiting system having a current limiting circuit (JP-A-7-184888) generally requires a time as much as several seconds due to the inertia force of the rotating motor until the rotation of the motor stops after the current to the motor is cut off. This system thus involves a potential risk that damage to a blood vessel or lumen may occur from the time an overload is applied to the driveshaft until the motor comes to a full stop. In addition, this construction relies upon an electrical cut-off and hence, is not well suited to eliminating the potential risk of an inadvertent actuation and is considered to have low reliability in stopping rotational drive.

On the other hand, the system described in JP-A-10-66696 that controls the drive torque of the rotational drive source by magnetic force and centrifugal force also involves a potential risk that damage to a blood vessel or lumen may increase because if an applied torque becomes smaller for one reason or another subsequent to the application of an overload to a driveshaft, the rotational drive source may be connected again to the rotational drive section to transmit drive force to the rotational drive section.

### SUMMARY

According to one aspect, an image diagnostic system comprises a probe positionable in a body cavity and configured to repeatedly transmit signals and acquire signals reflected from biotissue surrounding the body cavity during radial scanning, a scanner and pullback unit connected to the probe to rotate and axially move the probe during the radial scanning, and a torque limiter positioned to limit a torque load applied to the probe by the scanner and pullback unit, with the torque limiter comprising a shaft portion provided with a plurality of circumferentially arranged grooves which cause the shaft portion to break when the torque load applied by the scanner and pullback unit exceeds a predetermined load. In addition, a control unit is connected to the probe by way of a transmission line to produce digital data based on the acquired signals and to construct a tomographic image of the body cavity and the biotissue surrounding the body cavity on the basis of the digital data, and a display unit is connected to the control unit to display the tomographic image.

According to another aspect, an image diagnostic system comprises a probe positionable in a body cavity and configured to repeatedly transmit signals and acquire signals reflected from biotissue surrounding the body cavity during radial scanning, a control unit connected to the probe to produce digital data based on the acquired signals and to construct a tomographic image of the body cavity and the biotissue surrounding the body cavity on the basis of the digital data, and a display unit configured to display the tomographic image. The probe comprises a shaft transmitting a rotational drive force during the radial scanning by the probe, and a transmission line extending along the shaft to transmit the reflected signals to the control unit, with the shaft receiving the rotational drive force via a torque limiter. The torque limiter possesses a thickness which is non-uniform in a circumferential direction of the torque limiter at a part along a length of the torque limiter.

Another aspect involves a probe connectable to an image diagnostic apparatus and positionable in a body cavity, wherein the probe comprises an imaging core for transmitting signals and receiving reflected signals used by the image diagnostic apparatus to produce digital data for constructing a tomographic image of the body cavity and biotissue surrounding the body cavity, wherein the image core comprises a shaft configured to transmit rotational drive force to a distal portion of the imaging core, a transmission line extending along the shaft to transmit the reflected signals to the control unit, and a torque limiter positioned at a portion of the shaft to limit a torque load transmitted to the distal portion of the imaging core. The torque limiter possesses a thickness which is non-uniform in a circumferential direction of the torque limiter at a part along a length of the torque limiter.

In accordance with another aspect, a catheter comprises a sheath possessing a lumen, a shaft positioned in the lumen and configured to transmit a rotational drive force to a distal portion, and a torque limiter positioned at a proximal portion of the shaft to transmit the rotational drive force when the rotational drive force is less than a predetermined value, the torque limiter possessing a vulnerable portion along a circumferential direction that breaks upon application of a load torque equal to or greater than the predetermined value to prevent transmission of the torque load equal to or greater than the predetermined value to the distal portion.

With the system, probe and catheter disclosed herein, once an overload is applied to a distal end portion of a driveshaft, the drive is instantaneously and reliably cut off rotational drive force from the driveshaft.

### BRIEF DESCRIPTION OF THE DRAWING FIGURES

The foregoing and additional aspects of the disclosed probe and system will become more apparent from the following detailed description considered with reference to the accompanying drawing figures briefly described below.

FIG. 1 is a perspective view generally illustrating aspects and features of an IVUS imaging system according to a first embodiment disclosed herein.

FIG. 2 is a block diagram schematically illustrating additional aspects and features of the IVUS imaging system.

FIG. 3 is a plan view showing the construction of a control panel in the IVUS imaging system.

FIG. 4 is a perspective view of a catheter section in the IVUS imaging system.

FIG. 5 is a perspective view schematically illustrating the manner of sliding a driveshaft relative to a catheter sheath in the catheter section.

FIGS. 6A and 6B are perspective views in cross-section of a blood vessel and the catheter section inserted therein, illustrating movements of the catheter section during an intravascular ultrasound diagnosis.

FIG. 7 is a cross-sectional view of the distal end portion of the catheter section in the IVUS imaging system.

FIGS. 8A and 8B are cross-sectional views illustrating the internal construction of a driveshaft connector.

FIG. 9 is a cross-sectional view depicting details of a torque limiter.

FIGS. 10A through 10F are development views showing various examples of grooves formed in the torque limiter.

FIG. 11 is a perspective view depicting details of a torque limiting connector for an electric transmission line before breakage.

FIG. 12 is a perspective view depicting details of the torque limiting connector for the electric transmission line after break-off.

FIG. 13 is a fragmentary cross-sectional view showing a broken-off state of the torque limiter and torque-limiting connector when an overload has been applied to the distal end portion of a driveshaft.

FIGS. 14A and 14B are waveform diagrams illustrating the basic principle of optical coherence tomography (OCT).

FIG. 15 is a block diagram schematically illustrating the basic principle of an optical coherence tomography (OCT) system according to a second embodiment.

FIG. 16 is a block diagram depicting the features and aspects of the OCT imaging system.

FIG. 17 is a block diagram illustrating the basic principle of optical coherence tomography (OCT) making use of a wavelength swept light source.

FIG. 18 is a block diagram illustrating features and aspects of an optical coherence tomography (OCT) system according to a modification of the second embodiment of the present invention which makes use of a wavelength swept light source.

FIG. 19 is a cross-sectional view showing the construction of a distal end portion of a catheter section in the OCT imaging system of the second embodiment of the present invention or the OCT imaging system making use of a wavelength swept light source as a modification of the second embodiment.

FIGS. 20A and 20B are cross-sectional views showing the internal construction of a driveshaft connector without a scanner & pull-back unit connected thereto (FIG. 20A) and without the scanner & pull-back unit connected thereto (FIG. 20B).

FIG. 21 is a fragmentary cross-sectional view showing the construction of a general single-mode optical fiber.

FIG. 22 is a fragmentary cross-sectional view illustrating a prepared state of an end face of the optical fiber before setting it on an optical fiber splicing machine.

FIGS. 23A through 23C are fragmentary cross-sectional views showing the manner of fusion-splicing of the optical fibers by using the optical fiber splicing machine.

FIG. 24 is a fragmentary cross-sectional view illustrating the torque limiter and the fusion-spliced portion of the optical fiber in a state that they have broken off as a result of an application of an overload to a distal end portion of a driveshaft.

### DETAILED DESCRIPTION

### First Embodiment

### 1. General Overall Construction of IVUS Imaging System

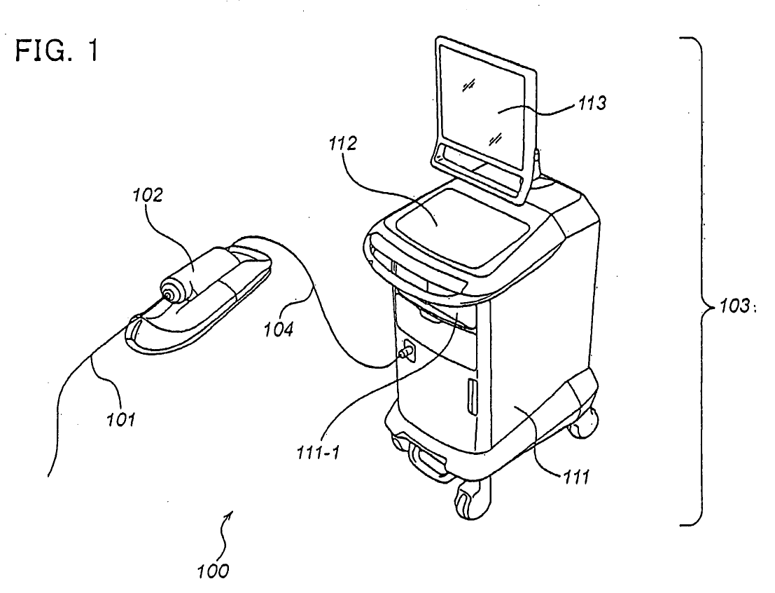

Referring to FIG. 1, an intravascular ultrasound (IVUS) imaging system (i.e., image diagnostic system) 100 according to one illustrated and disclosed embodiment includes a catheter section (i.e., probe) 101, a scanner & pull-back unit 102 and an operation control system 103. The scanner & pull-back unit 102 and the operation control system 103 are connected together via a signal line 104 and compose an image diagnostic apparatus.

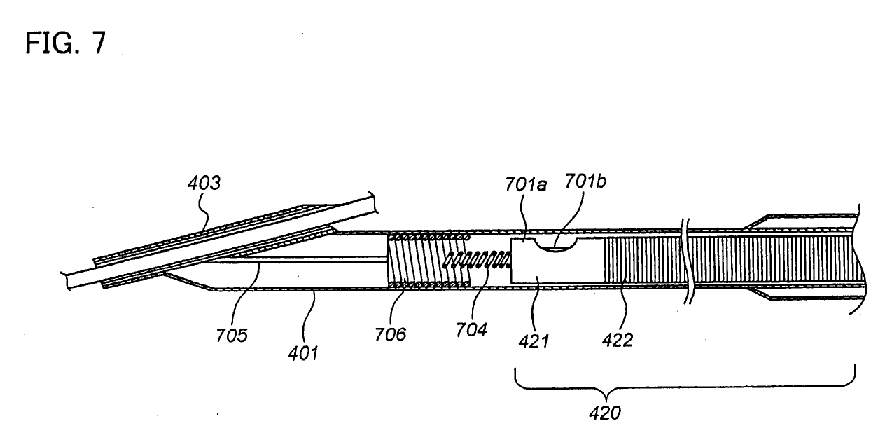

The catheter section 101 is adapted to be inserted directly into a blood vessel to measure internal conditions of the blood vessel by way of an ultrasonic transducer 701b which is shown in FIG. 7. The scanner & pull-back unit 102 controls movements of the ultrasonic transducer 701b within the catheter section 101.

The operation control system 103 operates to input various preset values upon performing an intravascular ultrasound diagnosis and to also process data acquired by a measurement and to display them as a tomographic image.

The operation control system 103 includes a main control unit 111 which performs processing of data acquired by a measurement and outputs the results of the processing, and a printer/DVD recorder 111-1 which prints the results of the processing in the main control unit 111 or records (i.e., stores) them as data.

The operation control system 103 also includes a control panel 112. Through the control panel 112, a user is able to input various values such as preset values. In addition, the operation control system 103 also includes an LCD monitor (i.e., display) 113, which displays the results of the processing in the main control unit 111.

### 2. Aspects and Features of IVUS Imaging System

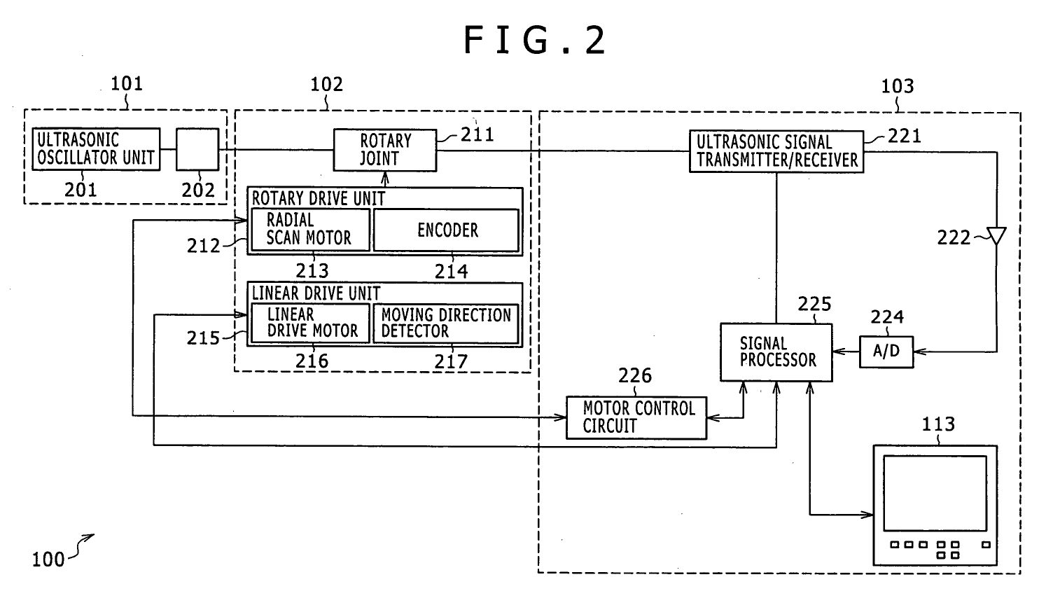

FIG. 2 schematically illustrates in more detail aspects and features of the IVUS imaging system 100 illustrated in FIG. 1. The distal end of the catheter section 101 is internally provided with an ultrasonic transducer unit 201. With the distal end of the catheter section 101 inserted within a blood vessel, the ultrasonic transducer unit 201, responsive to a pulse wave transmitted by an ultrasonic signal transmitter/receiver 221, transmits ultrasound in the direction of a section of the blood vessel, and receives the reflected signals (echoes) and transmits them as ultrasonic echo signals to the ultrasonic signal transmitter/receiver 221 via a connector 202 and a rotary joint 211.

The scanner & pull-back unit 102 includes the rotary joint 211, a rotary drive unit 212 and a linear drive unit 215. The ultrasonic transducer unit 201 within the catheter section 101 is rotatably mounted by the rotary joint 211, which connects a non-rotatable block and a rotatable block with each other, and is rotationally driven by a radial scan motor 213. Rotation of the ultrasonic transducer unit 201 in a circumferential direction within the blood vessel makes it possible to detect ultrasound echo signals required for the construction of a tomographic image of the blood vessel at the predetermined position within the blood vessel.

The operation of the radial scan motor 213 is controlled based on a control signal transmitted from a signal processor 225 via a motor control circuit 226. Further, each rotation angle of the radial scan motor 213 is detected by an encoder 214. Each output pulse outputted at the encoder 214 is inputted in the signal processor 225, and is used as a timing for the reading of signals to be displayed.

The scanner & pull-back unit 102 includes the linear drive unit 215 and, based on an instruction from the signal processor 225, specifies movements (axial movements) of the catheter section 101 in the direction of its insertion (in the directions toward and away from the distal direction within a body cavity). Axial movement is realized by operation of a linear drive motor 216 on the basis of a control signal from the signal processor 225. Further, the moving direction of the catheter section 101 in the axial direction (toward or away from the distal direction within the body cavity) is detected by a moving direction detector (i.e., detection unit) 217, and the result of the detection is inputted to the signal processor 225.

The ultrasonic signal transmitter/receiver 221 is provided with a transmission circuit and a reception circuit (not shown). Based on a control signal transmitted from the signal processor 225, the transmission circuit transmits a pulse wave to the ultrasonic transducer unit 201 in the catheter section 101.

The reception circuit, on the other hand, receives the signals based on ultrasonic echoes from the ultrasonic transducer unit 201 in the catheter section 101. The thus-received the signals are amplified by an amplifier 222.

At an A/D converter 224, the signals outputted from the amplifier 222 are sampled to produce digital data (ultrasound echo data) for one line.

Ultrasound echo data produced in line units at the A/D converter 224 are inputted into the signal processor 225. The signal processor 225 detects the ultrasound echo data, constructs tomographic images of the blood vessel at respective positions within the blood vessel, and outputs them at a predetermined frame rate to the LCD monitor 113.

### 3. Construction of Control Panel 112

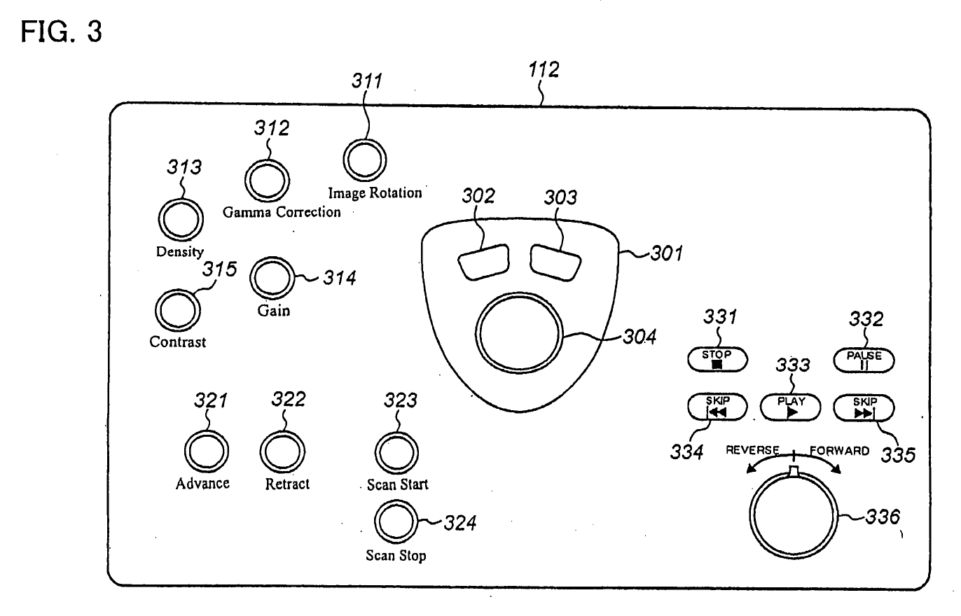

FIG. 3 is a plan view illustrating one example of the construction of the control panel 112. The control panel 112 includes an LCD monitor control unit 301 which is used to control various images to be displayed on the LCD monitor 113, a trackball 304 which is employed to control a pointer displayed on the LCD monitor 113, and left and right click buttons 302, 303, respectively. The setting of various conditions (for example, rotational speed) in a diagnosis is effected via the LCD monitor control unit 301.

The control panel 112 also includes setting dials (i.e., knobs) 311-315 for setting correction values upon processing ultrasound echo data at the signal processor 225, an image rotation setting dial 311 which sets the direction upon rotating a tomographic image produced based on inputted ultrasound echo data, and a gamma correction setting dial 312 which is used to finely adjust the gamma value to perform color matching. In addition, a density setting dial 313 is provided to adjust the density of a tomographic image to be displayed, a gain setting dial 314 permits adjustment of the gain for ultrasound echo data to be inputted and a contrast setting dial 315 permits adjustment of the contrast of a tomographic image to be displayed.

The control panel 112 further includes buttons 321-323 used during radial scanning by the ultrasonic transducer unit 201 in the catheter section 101, an advance button 321 and a retract button 322. While the advance button 321 is being pressed, the linear drive motor 216 continues to operate so that the ultrasonic transducer unit 201 in the catheter section 101 moves toward the periphery within the body cavity, and when the pressing is cancelled the movement stops). While the retract button 322 is being pressed, the linear drive motor 216 continues to operate so that the ultrasonic transducer unit 201 in the catheter section 101 moves away from the periphery within the body cavity, and when the pressing is cancelled the movement stops.

The control panel 112 is also equipped with a scan start button 323 and a scan stop button 324. When the scan start button 323 is pressed the radial scan motor 213 is operated to drive the ultrasonic transducer unit 201 in the catheter section 101 at a predetermined rotational speed, and when the scan stop button 324 is pressed the rotating ultrasonic transducer unit 201 is stopped.

Additionally, several buttons 331-335 and a dial 336 are provided and are to be used to display stored tomographic images on the LCD monitor 113, a PLAY button 333 is used to display stored tomographic images at a predetermined frame rate on the LCD monitor 113, a STOP button 331 is used to stop the display of tomographic images, and a PAUSE button 332 is used to temporarily stop tomographic images under display at a predetermined frame rate.

The control panel 112 further includes a skip-up button 334 which is used to jump up to a tomographic image at a predetermined position from a tomographic image currently under display (to a tomographic image at a backward position from a tomographic image currently under display), a skip-down button 335 which is used to jump down to a tomographic image at a predetermined position from a tomographic image currently under display (to a tomographic image at a forward position from a tomographic image currently under display), and a quick forward/reverse dial 336 which. When the quick forward/reverse dial 336 is rotated clockwise tomographic images are quickly displayed forward at a predetermined frame rate, and when the quick forward/reverse dial 336 is rotated counterclockwise tomographic images are displayed backward at a predetermined frame rate.

With the above-described control panel 112, radial scanning by the ultrasonic transducer unit 201 is realized by operating/controlling different control buttons and dials to perform linear movements and rotational movements. The system, apparatus and method disclosed herein is not, however, limited to such a control panel. For example, an extra control button may be arranged to directly achieve radial scanning. On the other hand, each linear movement may be achieve by pressing the corresponding control button or by directly moving the scanner & pull-back unit 102 forward or backward by hand. Even in such a modification, the moving direction of each linear movement can be similarly detected by the moving direction detector 217.

### 4. Construction of Catheter Section

### 4.1 Overall Construction of Catheter Section

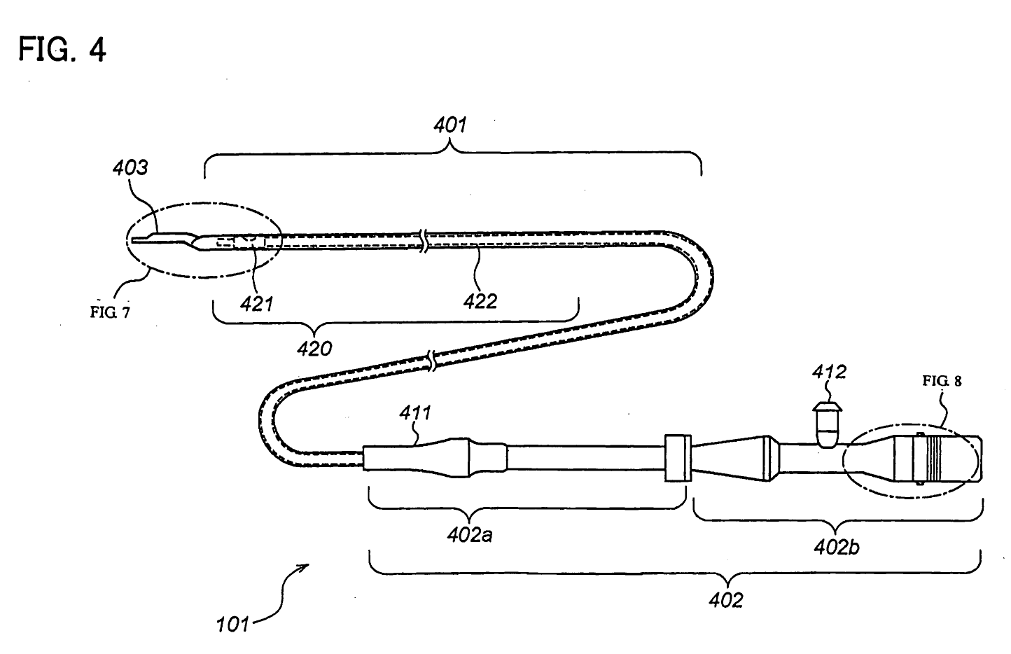

The overall general construction of the catheter section 101 is illustrated in FIG. 4. The catheter section 101 is constructed as an elongated catheter sheath 401 adapted to be inserted into a blood vessel and a connector 402, not intended to be inserted into the blood vessel, that is arranged on the side of the user's hand to permit handling and operation by the user. A guidewire lumen 403 is provided at the distal end of the catheter sheath. Within the catheter sheath 401 is a lumen which continuously extends from a connecting portion with the guidewire lumen 403 to a connecting portion with the connector 402.

Through the lumen of the catheter sheath 401, an imaging core 420 extends over substantially the entire length of the catheter sheath 401. The imaging core 420 is provided with an ultrasonic transducer unit 421 for transmitting and receiving ultrasound and also with the hollow driveshaft 422 for transmitting drive force to rotate the ultrasonic transducer unit 421.

The connector 402 is composed of a sheath connector 402a and a driveshaft connector 402b. The sheath connector 402a is constructed integrally with a proximal end of the catheter sheath 401. The driveshaft connector 402b is arranged on a proximal end of the driveshaft 422 to rotatably hold the drive shaft 422.

An anti-kink protector 411 is arranged at the distal end of the sheath connector 402a or at the boundary portion between the proximal end portion of the sheath connector 402a and the catheter sheath 401. This anti-kink protector 411 makes it possible to maintain a predetermined degree of stiffness, thereby helping to prevent any short tight twist or curl which might otherwise be caused by a sudden change in torque. The driveshaft connector 402b is provided with an injection port 412 to which a syringe (not illustrated) or the like can be attached to fill up the lumen of the catheter sheath 401 in its entirety with an ultrasound transmission fluid. The proximal end of the driveshaft connector 402b is constructed to be connected to the scanner & pull-back unit 102.

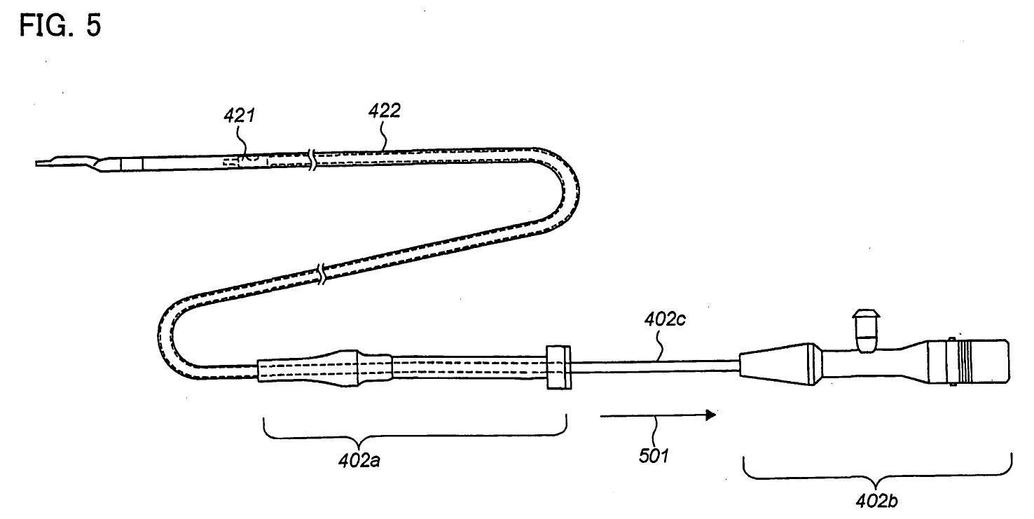

FIG. 5 schematically illustrates the manner in which the driveshaft 422 is slidably pulled back relative to the catheter sheath 401. As illustrated, the sliding of the driveshaft connector 402b toward its proximal end (in the direction of arrow 501) by the scanner & pull-back unit 102 with the sheath connector 402a being held fixed causes the driveshaft 422, which is accommodated within the driveshaft connector 402b, and the ultrasonic transducer unit 421, which is fixedly secured on the distal end of the driveshaft 422, to slide in the axial direction. This axial sliding may be effected either manually by the user or by an electrical drive. On the distal end side of the driveshaft connector 402b, a protecting inner tube 402c is arranged to avoid exposure of the driveshaft 422 which rotates at a high speed.

### 4.2 Operation of Catheter Section 101 Upon Intravascular Ultrasound Diagnosis

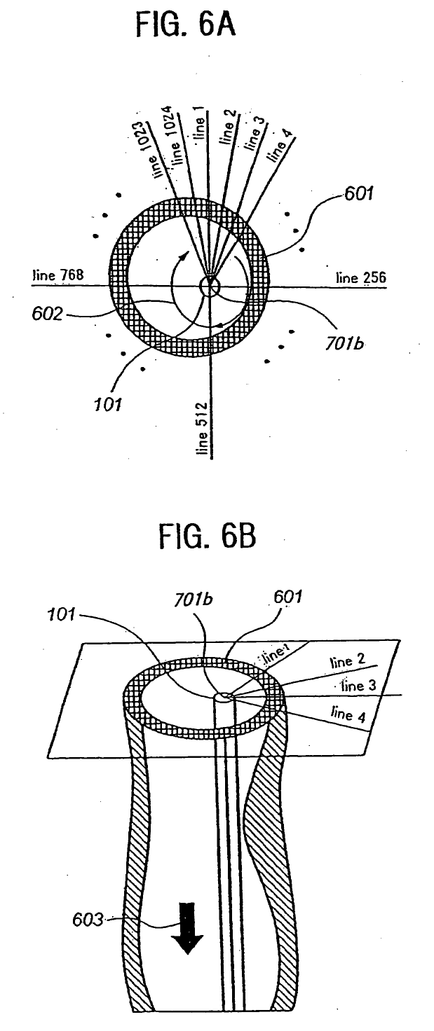

FIGS. 6A and 6B schematically illustrate movements of the catheter section 101 during an intravascular ultrasound (IVUS) diagnosis. FIGS. 6A and 6B illustrate, in cross-section and perspective view respectively, a blood vessel with the catheter section 101 inserted therein.

FIG. 6A illustrates a section of the blood vessel 601 in which the catheter section 101 is inserted. As described above, the ultrasonic transducer 701b is internally mounted at the distal end of the catheter section 101, and is rotated in the direction of arrow 602 by the radial scan motor 213.

From the ultrasonic transducer 701b, the transmission/reception of ultrasound is performed at respective rotation angles. Lines 1, 2, . . . , 1024 indicate the transmitting directions of ultrasound at the respective rotation angles. In this embodiment, 1,024 transmissions/receptions are intermittently performed while the ultrasonic transducer 701b rotates over 360 degrees in a predetermined blood vessel section (601). The number of transmissions/receptions of ultrasound during a 360-degree rotation is not limited specifically to 1,024, but can be set as desired. The scanning that is repeated with the transmission/reception of a signal while rotating the ultrasonic transducer 701b as described above is generally called “radial scan” or “radial scanning”.

Such transmissions/receptions of ultrasound are performed while advancing the catheter section through the blood vessel in the direction of arrow 603 shown in FIG. 6B.

### 4.3 Construction of Distal End Portion of Catheter Section

FIG. 7 illustrates in more detail the distal end portion of the catheter section 101. The ultrasonic transducer unit 421 is comprised of an ultrasonic transducer 701b and a housing 701a in which the ultrasonic transducer 701b is held. Ultrasound is transmitted from the ultrasonic transducer 701b toward surrounding biotissue of a body cavity, and reflected waves from the surrounding biotissue of the body cavity are received at the ultrasonic transducer 701b.

The driveshaft 422 is a hollow shaft constructed in the form of a coil, accommodates an electric transmission line therein, and extends from the ultrasonic transducer 701b to the connector 402.

The ultrasonic transducer 701b possesses a rectangular or circular shape, and is formed by depositing electrodes on opposite sides of a piezoelectric member made of PZT or the like. The ultrasonic transducer 701b is arranged to assume a position around a central axis of rotation to prevent the driveshaft 422 from causing rotational fluctuations.

The housing 701a is in the form of a short cylindrical tube provided at a part thereof with a cut-off portion. Examples of materials forming the housing 701a include metal or hard resin. Examples of methods of forming include machining such as cutting, laser machining or pressing may be applied to a tubular material to form the cut-off portion, or the desired shape may be directly obtained by injection molding, MIM (metal injection molding) or the like. The proximal end side of the housing 701a is connected with the driveshaft 422. On the distal end side of the housing 701a, a resilient member 704 in the form of a short coil is arranged.

The resilient member 704 is a coil-shaped wire which can be produced by forming a stainless steel wire into a coiled shape. The arrangement of the resilient member 704 on the distal end side of the housing 701a provides the imaging core 420 with improved stability upon rotation. Gold plating can be applied to a surface of the resilient member 704 or housing 701a. As gold is a metal having high x-ray opacity, the gold plating can permit visualization of the resilient member 704 in an image taken by an x-ray imaging system when the catheter sheath 401 is inserted into a body cavity. As a result, the user can easily ascertain the position of the ultrasonic transducer 701b.

A discharge channel 705 is arranged at a boundary portion between the distal end portion of the catheter sheath 401 and the guidewire lumen 403, the discharge channel 705 is arranged to discharge the ultrasound transmission fluid injected in the priming work.

A reinforcement coil 706 is arranged to avoid kinking of the distal end portion of the catheter sheath 401.

The guidewire lumen 403 has a bore into which the guidewire is adapted to be inserted. The guidewire is inserted beforehand in a body cavity, and is utilized to guide the catheter sheath 401 to a diseased part.

The driveshaft 422 is constructed of a multiple or multilayer, tight coil or the like having properties such that it can rotate and slide relative to the catheter sheath 401. The driveshaft 422 is flexible and can smoothly transmit rotation. The multiple or multilayer, tight coil or the like may be made, for example, of a wire of a metal such as stainless steel.

### 4.4 Construction of Driveshaft Connector 402b

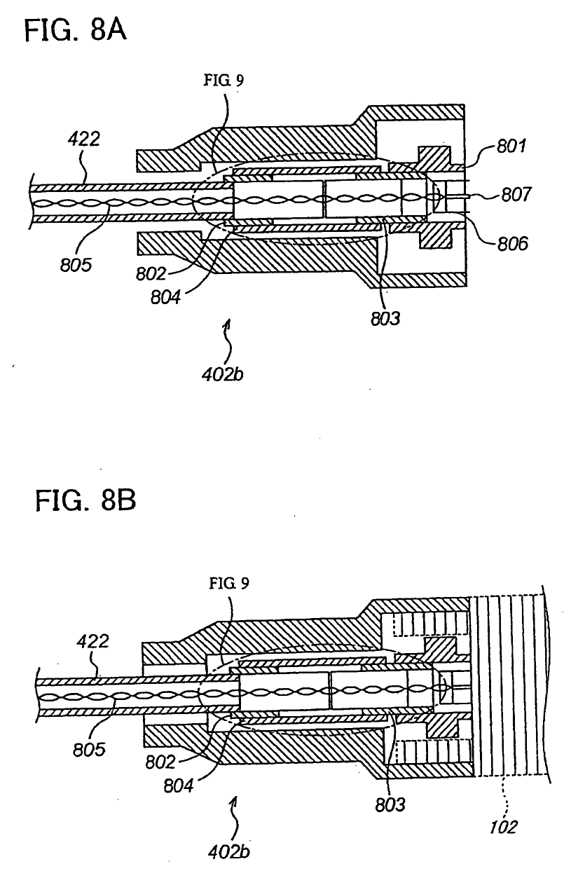

The internal construction of the driveshaft connector 402b is shown in FIGS. 8A and 8B, with FIG. 8A illustrating the driveshaft connector 402b without the scanner & pull-back unit 102 and FIG. 8B illustrating the driveshaft connector 402b connected to the scanner & pull-back unit 102.

As depicted in FIGS. 8A and 8B, the driveshaft 422 and a connector 801 are coupled with each other at a torque limiter 804 via a distal-end-side pipe 802 and a user-side pipe 803.

An electric transmission line 805 through which the ultrasonic transducer 701b is energized extends through the lumen within the driveshaft 422, and is divided into a signal electrode 807 and another signal electrode 806 at the connector 801. In this illustrated embodiment, the electric transmission line 805 is a twisted-pair line, but it is to be understood that the electric transmission line 805 can take forms others than the twisted-pair line, for example a coaxial line.

A portion of the electric transmission line 805 inside the torque limiter 804 is formed as a torque-limiting connector (the details of which will be described below), thereby providing a mechanism in which rotational drive force transmitted form the scanner & pull-back unit 102 can be cut off when a load torque of a predetermined value or greater is applied.

### 4.5 Construction of Torque Limiter 804

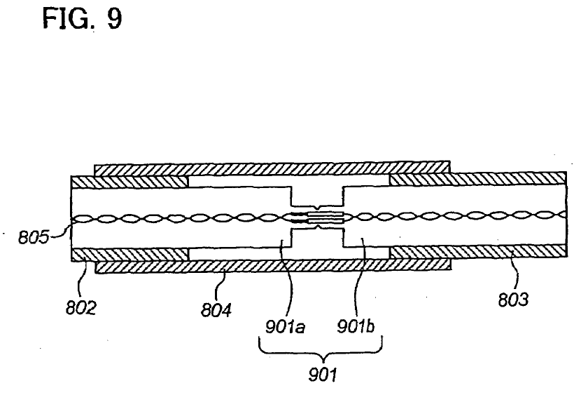

Additional details associated with the torque limiter 804 are shown in FIG. 9. The torque limiter 804 includes, at a part thereof, one or more grooves formed in a circumferential direction. As described below in more detail, the one or more grooves can take various forms, for example slots or slits which provide a portion of non-uniform thickness in a circumferential direction. The groove(s) form a vulnerable portion at the torque limiter 804, the torque limiter 804 is provided with a mechanism by which the rotational drive force to be transmitted from the scanner & pull-back unit 102 to the driveshaft 422 is cut off by the destruction of the torque limiter 804 when a load torque of the predetermined value or greater is applied to the distal end portion of the driveshaft 422.

In this embodiment, the cut-off torque of the torque limiter 804 is, from a practical standpoint, preferably 0.1 to 5 mN·m, more preferably 0.5 to 2 mN·m. The material forming the torque limiter 804 itself is preferably a resin material, paper or pulp material, or inorganic material, with a material suited for a process that forms grooves with good reproducibility and high preciseness, being more preferred. An excimer laser can be used for forming grooves with good reproducibility and relatively high precision.

Within the torque limiter 804, the electric transmission line 805 is connected by a torque-limiting connector 901. The torque-limiting connector 901 includes a distal-end-side portion 901a and a user-side portion 901b, each of which has a semi-cylindrical shape and is made of an insulating material.

### 4.5.1 Manufacturing Process for Forming the Torque Limiter 804

Set forth below is a description of a process for manufacturing the torque limiter 804. This process, disclosed by way of example, is an excimer laser processing. An excimer laser is a pulse-oscillating, UV-wavelength gas laser. The mixed gas used in this process is a mixed gas of a rare gas and halogen gas. These gases are diluted with a buffer gas such as He or Ne to raise the total pressure to 4 atm or so. The resulting gas mixture is excited by a discharge to oscillate a laser beam having a pulse width of 10 ns or smaller. It is KrF (wavelength: 248 nm) that is commonly used as a mixed gas in an excimer laser.

An excimer laser machining process is generally referred to as an ablation process. It is a process involving gasifying a polymer material or dividing the polymer material into microparticles by causing the polymer material to instantaneously absorb energy greater than its intermolecular bonding force and breaking its intermolecular bonds. As a consequence, micro-machining with minimized thermal effects can be performed. It is to be noted that micro-machining of 1 μm level is theoretically feasible because an excimer laser permits easy beam focusing.

Examples of other lasers include a high-power harmonic Q switch Nd:YAG laser (wavelength 266 nm), which can oscillate ultraviolet rays, and a pulse-oscillating carbon dioxide laser. These lasers can form grooves similarly as in the case of the excimer laser, although they are somewhat inferior in machining precision.

An example of another method for forming grooves is a method that makes use of a cutter, such as a microcutter. With this method, however, it is a little difficult to form grooves with good reproducibility and high precision because the sharpness of a cutting blade tends to deteriorate.

Polyimide can be mentioned as a representative polymer material for the torque limiter, which permits good micro-machining by an excimer laser. Polyimide is an engineering plastic, and owing to its high Young's modulus, still retains practical strength even when formed into the shape of a thin-walled tube. Such a thin-walled tube is convenient to perform the machining of fine through-grooves by an excimer laser.

### 4.5.2 Example 1 of the Torque Limiter 804

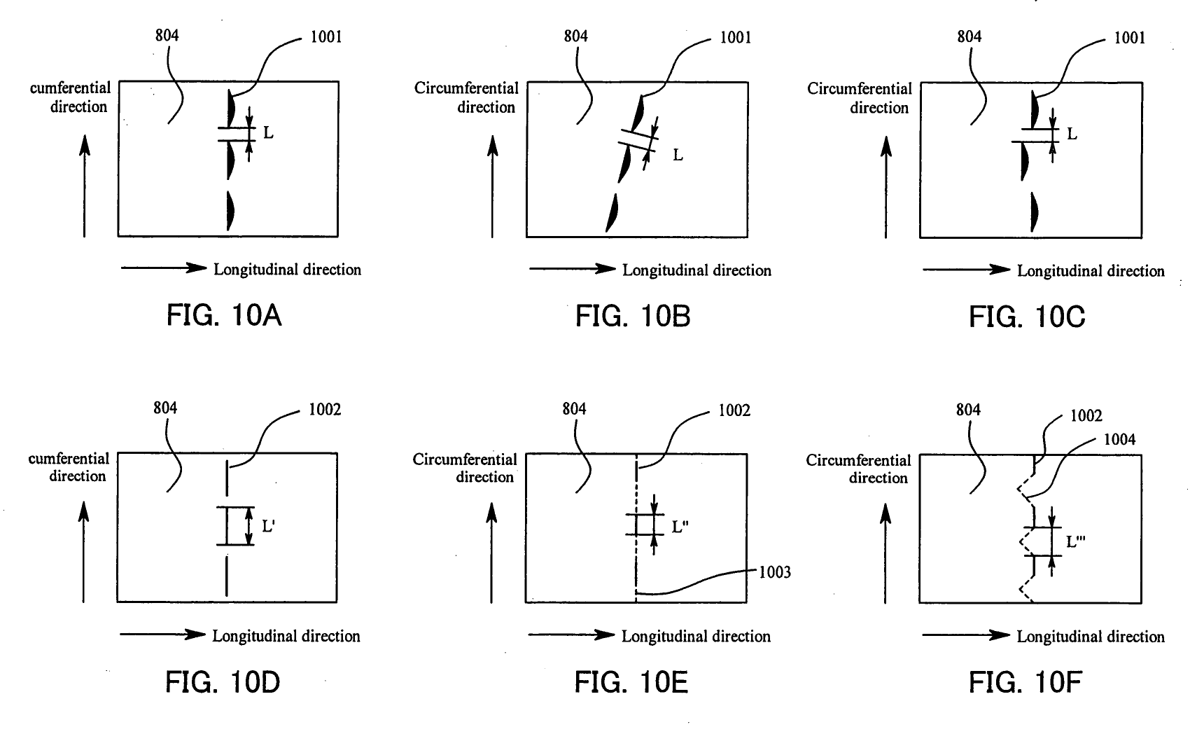

FIGS. 10A, 10B and 10C are development views of the torque limiter 804, which illustrate one example of the shape of grooves formed in the torque limiter 804. In FIG. 10A, the grooves formed in the torque limiter 804 are trapezoidal slots, each of which can be formed by patterning, on the surface of the torque limiter 804, a small beam spot of the excimer laser in a trapezoidal shape in accordance with a program inputted beforehand. These plural trapezoidal slots 1001 are formed in either a through state or a non-through state, and are aligned in a circumferential direction at a part of the torque limiter 804 along the length of the torque limiter 804. That is, the thickness of the torque limiter 804 is formed so as to be non-uniform in the circumferential direction at the part of the torque limiter 804 along the length of the torque limiter 804.

As shown in FIGS. 10B and 10C, the slots may alternatively be formed in a spiral pattern or may be alternately formed. Instead of the trapezoidal shape, the slots may be formed to possess a rhombic, isosceles triangular, elliptical or racetrack shape or may be formed in any other desired formable shape. The beam spot size of the excimer laser may preferably be set as small as possible. As a practical size, a diameter of from 0.01 to 0.02 μm is preferred. The beam spot may be a circular, rectangular or triangular shape or any other desired formable shape.

In this embodiment, it is preferred to have at least three slots, because with two or fewer slot(s), stress tends to concentrate at hinge portions (portions where no slots have been machined) so that the torque limiter is susceptible to breakage upon assembly. In FIGS. 10A through 10C, the length of each slot interval L depends on the material of the torque limiter 804 and the number of slots. When the number of the slots is 3, for example, the slot interval L may preferably be from 0.2 to 0.3 mm.

### 4.5.3 Example 2 of the Torque Limiter 804

FIGS. 10D, 10E and 10F are development views of the torque limiter 804, illustrating another example of the shape of the grooves formed in the torque limiter 804. In FIG. 10D, the grooves formed in the torque limiter 804 are in the form of slits. An excimer laser beam spot is moved, as is, in a circumferential direction to form intermittent through-slits 1002. The length L′ of each through-slit may preferably be from 0.2 to 0.3 mm. In FIG. 10E, through-slits 1002 and non-through-slits (broken dashes) 1003 are combined. To set the cut-off torque at the same level as in FIG. 10D, the length L″ of each through-slit 1002 can be set shorter, thereby bringing about an advantage that the torque limiter 804 can be provided with improved breakage resistance during assembly. In FIG. 10F, through-slits 1002 and turned V-shaped, non-through-slits (broken lines) 1004 are formed in combination. To set the cut-off torque at the same level as in FIGS. 10D and 10E, the length L′″ of each through-slit 1002 can be set between L′ and L″ while bringing about an advantage that the torque limiter 804 can be provided with further improved breakage resistance during assembly. Instead of the turned V shape, the non-through slits 1004 may be formed in a semicircular or arcuate shape or in any other desired shape or arrangement. These slits can all be non-through slits.

### 4.5.4 Construction of Torque-Limiting Connector 901

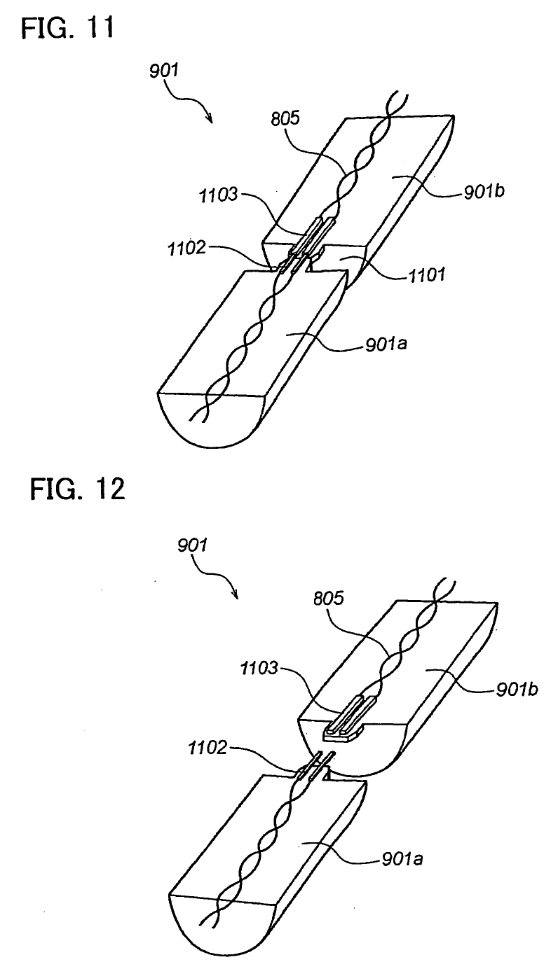

FIGS. 11 and 12 illustrate details of the torque-limiting connector 901 for the electric transmission line 805. As illustrated in FIG. 11, the torque-limiting connector 901 is composed of a semi-cylindrical, insulating member and includes a constricted portion 1101 at a part thereof, and therefore, is constructed such that it breaks at a load torque of a predetermined value or greater. The electric transmission line 805 of the twisted-pair line is constructed in a disconnectable manner at male terminals 1102 and female terminals 1103, and the respective terminals are connected together at the constricted part 1101.

The distal-end-side portion 901a and user-side portion 901b as semi-cylindrical portions of the torque-limiting connector 901 are fixedly adhered to the inner walls of the distal-end-side pipe 802 and user-side pipe 803, respectively. The material of the torque-limiting connector 901 is preferably a resin material, more preferably a resin material having good injection moldability and good adhesion property with various adhesives.

### 4.6 Operations of the Torque Limiter 804 and the Torque-Limiting Connector 901

When a load toque of the predetermined value or greater is applied to the distal end portion of the driveshaft 422 while the driveshaft 422 is being rotated at a high speed with the electric transmission line 805 connected through the terminals on the torque-limiting connector 901, the torque limiter 804 is broken to cut off the rotational drive force from the driveshaft 422 and at the same time, the torque-limiting connector 901 is also broken. As a result, the terminals of the electric transmission line 805 are disconnected as shown in FIG. 12 so that the electric transmission line 805 is also cut off. According to this embodiment, it is thus possible to instantaneously cut off the rotational drive force transmitted from the scanner & pull-back unit 102.

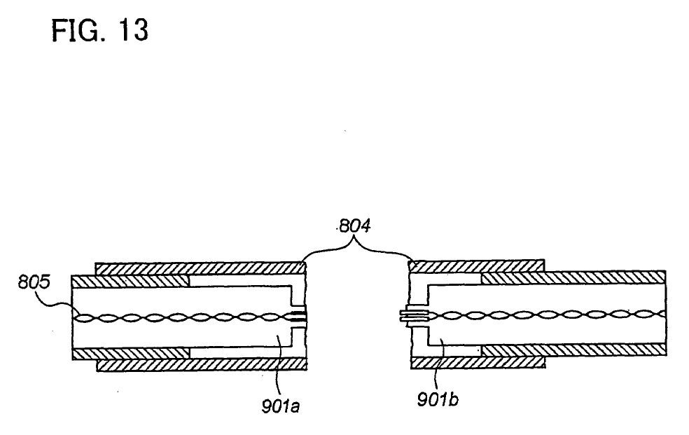

FIG. 13 is a fragmentary cross-sectional view illustrating a state in which the torque limiter 804 and torque-limiting connector 901 have been broken as a result of an application of an overload to the distal end portion of the driveshaft 422. When a load torque of the predetermined value or greater is applied to the distal end portion of the driveshaft 422, the rotational drive force from the scanner & pull-back unit 102 to the driveshaft 422 is cut off, and at the same time, the torque-limiting connector 901 breaks off.

The IVUS imaging system described above makes it possible to instantaneously cut off the rotational drive force from the driveshaft to thus reliably stop the rotation of the probe when an overload is applied to the distal end portion of the driveshaft, because the torque limiter and torque-limiting connector instantaneously break off at the time of application of the overload.

Although the torque limiter 804 is broken as a result of the generation of torque of the predetermined value or greater in the system 100 described above, the system 100 may incrementally or additionally comprise a function that the radial scan motor 213 is stopped automatically by the motor control circuit 236 when the motor control circuit 236 detects a torque which is the same as the predetermined value or a second predetermined value a little smaller than the predetermined value. The motor control circuit 236 is able to detect the value of the torque, for example, from the number of rotations per voltage.

### Second Embodiment

The description above describes the probe (catheter section) in the IVUS imaging system. However, the disclosure here is not specifically limited to IVUS imaging systems, but rather has useful application to other image diagnostic systems. The following describes application of the disclosure here to probes of an optical coherence tomography (OCT) imaging system and an OCT imaging system making use of a wavelength swept light source as a modification of the first-mentioned OCT imaging system.

### 1. Measurement Principle of OCT Imaging System

The measurement principle of an OCT imaging system will first be briefly described. Because light is electromagnetic radiation, it generally has the property that beams of light interfere with each other when they are superimposed. The interference property that defines whether light interferes readily or hardly is called “coherence”, and in general OCT imaging systems, low-coherence light of low interference property is used.

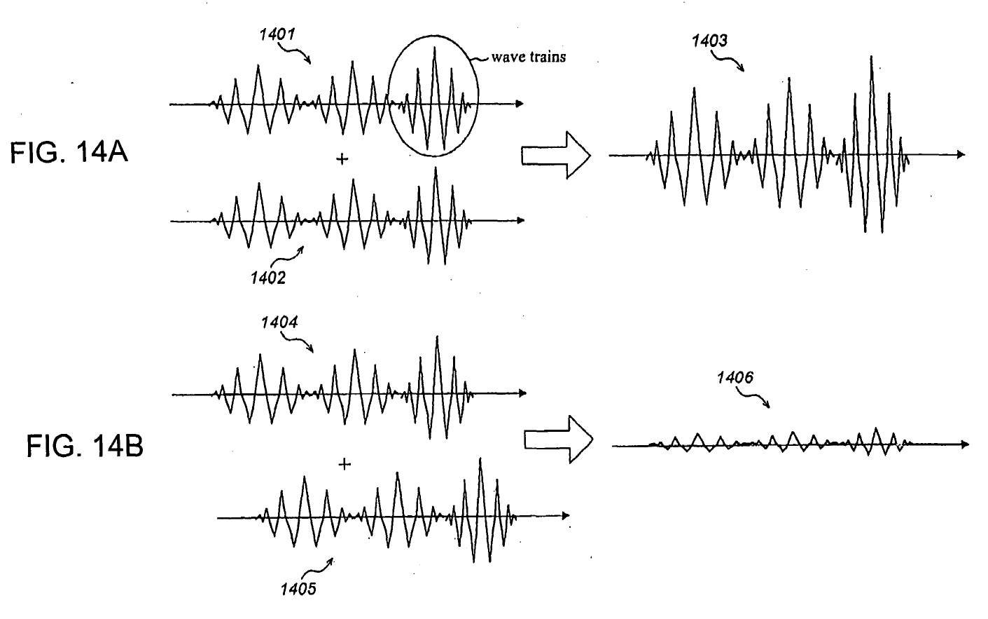

When time is plotted along the abscissa and the electric field is plotted along the coordinate, low-coherence light becomes random signals as indicated at 1401 and 1402 in FIG. 14A. Individual peaks in the figure are called “wave trains”, and have their own, mutually-independent phases and amplitudes. When the same wave trains (1401 and 1402) overlap with each other as in FIG. 14A, they interfere with each other to intensify each other (see 1403). On the other hand, when there is a slight delay in time between wave trains (1404 and 1405 in FIG. 14B), they cancel each other so that no interference light is observed as shown at 1406 in FIG. 14B.

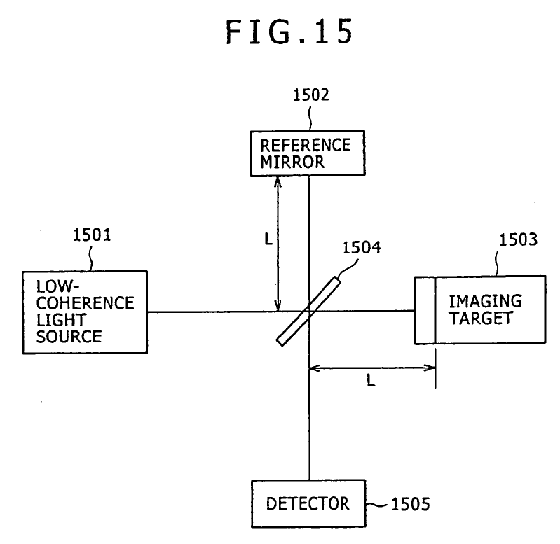

The OCT imaging system makes use of such properties, and the basic principle of the system is illustrated in FIG. 15. As shown in the figure, light emitted from a low-coherence light source 1501 is split at a beam splitter 1504 between a reference optical path and a sample optical path. The resulting light beam in the reference optical path is then directed toward a reference mirror 1502. Further the resulting light beam in the sample optical path is then directed toward an imaging target 1503. At this time, reflected light which is returning from the path of the imaging target includes light reflected on the surface of the imaging target, light reflected at shallow points in the imaging target, and light reflected at deep points in the imaging target.

As the incident light is low-coherence light, the reflected light on which interference can be observed is, however, only the reflected light from a reflection surface located at a position apart by a distance of L+ΔL/2 from the beam splitter 1504, where L represents the distance from the beam splitter 1504 to the reference mirror 1502, and ΔL represents a coherence length.

By changing the distance from the beam splitter 1504 to the reference mirror 1502, it is possible to selectively detect at a detector 1505 only reflected light from a reflection surface, which corresponds to the thus-changed distance, in the imaging target. A tomographic image can then be constructed by visualizing internal structural information of the imaging target on the basis of the intensities of reflected light beams corresponding to such respective distances.

### 2. General Overall Construction of OCT Imaging System

The general overall construction of the OCT imaging system is similar to that of the IVUS imaging system described above in the first embodiment as shown in FIG. 1. A description of the general overall construction is thus not repeated.

### 3. Aspects and Features of OCT Imaging System

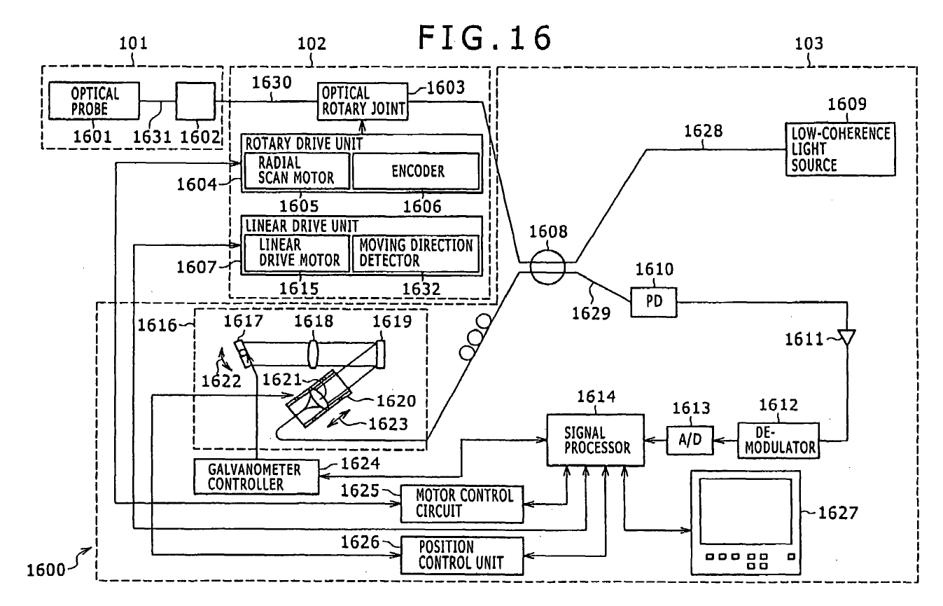

FIG. 16 illustrates features and aspects associated with the OCT imaging system (image diagnostic system) 1600. The system includes a low-coherence light source 1609 such as an ultra-high intensity, light emitting diode. The low-coherence light source 1609 has a wavelength around 1,310 nm, and outputs low-coherence light showing interference property only in such a short distance range that its coherence length approximately ranges from several micrometers to over ten micrometers.

When the light is split into two and the resulting beams of light are combined back, the combined light is, therefore, detected as coherent light when the difference between the two optical path lengths from the splitting point to the combining point falls within a short distance range around 17 μm, but no coherent light is detected when the difference in optical path length is greater than the above-described range.

The light from the low-coherence light source 1609 impinges on a proximal end face of a first single mode fiber 1628, and is transmitted toward its distal end face. At an optical coupler 1608 arranged midway along the first single mode fiber 1628, the first single mode fiber 1628 is optically coupled with a second single mode fiber 1629. Therefore, the light transmitted through the first single mode fiber 1628 is split into two by the optical coupler 1608 and the resulting two beams of light are transmitted further.

On the more distal end side of the first single mode fiber 1628 than the optical coupler 1608, an optical rotary joint 1603 is arranged to connect a non-rotatable block and a rotatable block with each other such that light can be transmitted.

Further, an optical-probe connector 1602 is detachably connected to a distal end of a third single mode fiber 1630 in the optical rotary joint 1603. Via the connector 1602, the light from the low-coherence light source 1609 is transmitted to a fourth single mode fiber 1631, which is inserted in an optical probe 1601 and is rotationally drivable.

The transmitted light is irradiated from a distal end side of the optical probe 1601 toward a surrounding biotissue of a body cavity while performing radial scanning. A portion of reflected light scattered on a surface or interior of the biotissue is collected by the optical probe 1601, and returns to the side of the first single mode fiber 1628 through the reverse optical path. A portion of the thus-collected, reflected light is transferred by the optical coupler 1608 to the side of the second single mode fiber 1629, and is introduced into a photodetector (for example, photodiode 1610) from an end of the second single mode fiber 1629.

It is to be noted that the rotatable block side of the optical rotary joint 1603 is rotationally driven by a radial scan motor 1605 of a rotary drive unit 1604. Further, rotation angles of the radial scan motor 1605 are detected by an encoder 1606. The optical rotary joint 1603 is provided with a linear drive unit 1607 that, based on an instruction from a signal processor 1614, controls movement (axial movement) of the catheter section 101 in the direction of its insertion (toward or away from a distally within a body cavity). An axial movement of the catheter section 101 is realized by an operation of a linear drive motor 1615 on the basis of a control signal from the signal processor 1614. Further, the moving direction of the catheter section 101 in its axial movement (toward or away from the distally within the body cavity) is detected by a moving direction detector 1632, and the result of the detection is inputted to the signal processor 1614.

On the side of a more distal end of the second single mode fiber 1629 than the optical coupler 1608, an optical path length (OPL) varying mechanism 1616 is arranged to vary the optical path length of reference light.

This OPL varying mechanism 1616 is provided with a first OPL varying means for varying the optical path length, which corresponds to the examinable range in the direction of the depth of the biotissue, at high speed and also with a second OPL varying means for varying the optical path length by a length equivalent to a variation in the length of a new optical probe to absorb or adjust the variation when the new optical probe is used as a replacement since the probe used for inserting the blood vessel of human is generally disposable.

Opposing a distal end of the second single mode fiber 1629, a grating (diffraction grating) 1619 is arranged via a collimator lens 1621 which is mounted together with the distal end of the second single mode fiber 1629 on a single axis stage 1620 and is movable in the direction indicated by arrow 1623. Further, a galvanometer mirror 1617 which is rotatable over small angles is mounted as the first OPL varying means via the grating 1619 and an associated lens 1618. This galvanometer mirror 1617 is rotated at high speed in the direction of arrow 1622 by a galvanometer controller 1624.

The galvanometer mirror 1617 serves to reflect light by its mirror, and functions as a reference mirror. The galvanometer mirror 1617 is constructed such that its mirror mounted on a movable part of its galvanometer is rotated at high speed by applying an a.c. drive signal to the galvanometer.

More specifically, by applying a drive signal to the galvanometer from the galvanometer controller 1624 and rotating the galvanometer at high speed in the direction of arrow 1622 with the drive signal, the optical path length of reference light is varied at high speed by an optical path length equivalent to an examinable range in the direction of the depth of the biotissue. A single cycle of variations in optical path length becomes a cycle that acquires interference light data for a single line.

On the other hand, the single axis stage 1620 forms the second OPL varying means having a variable OPL range just enough to absorb a variation in the optical path length of a new optical probe when the optical probe 1601 is replaced by the new optical probe. In addition, the single axis stage 1620 is also equipped with a function as an adjustment means for adjusting an offset. Even when the distal end of the optical probe 1601 is not in close contact with a surface of the biotissue, for example, the optical probe can still be set in such a state as interfering from a position on the surface of the biotissue by slightly varying the optical path length with the single axis stage 1620.

The light varied in optical path length by the OPL varying mechanism 1616 is combined with the light, which has escaped from the side of the first single mode fiber 1628, at the optical coupler 1608 arranged midway along the second single mode fiber 1629, and the combined light is received at the photodiode 1610.

The light received at the photodiode 1610 is photoelectrically converted, amplified by an amplifier 1611, and then inputted into a demodulator 1612. At the demodulator 1612, demodulation processing is performed to extract only the signal portion of the interfered light, and the output of the demodulator 1612 is inputted into an A/D converter 1613.

At the A/D converter 1613, interference light signals are sampled as much as for 200 points to produce digital data (interference light data) for one line. The sampling frequency is a value obtained by dividing with 200 the time required for a single scan of the optical path length.

The interference light data in line unit, which have been produced at the A/D converter 1613, are inputted into the signal processor 1614. At this signal processor 1614, the interference light data in the direction of the depth are converted into video signals to constitute tomographic images at respective positions in the blood vessel. These tomographic images are then outputted at a predetermined frame rate to an LCD monitor 1627.

It is to be noted that the signal processor 1614 is connected with a position control unit 1626. The signal processor 1614 performs control of the position of the single axis stage 1620 via the position control unit 1626. In addition, the signal processor 1614 is also connected with a motor control circuit 1625 to control rotational drive by the radial scan motor 1605.

Further, the signal processor 1614 is also connected with the galvanometer controller 1624 which controls the scanning of the optical path length of the reference mirror (galvanometer mirror). The galvanometer controller 1624 outputs a drive signal to the signal processor 1614, and based on this drive signal, the motor control circuit 1625 is synchronized with the galvanometer controller 1624.

### 4. Measurement Principle of the OCT Imaging System Making Use of a Wavelength Swept Light Source

Initially, a brief description is set forth of the measurement principle of an OCT imaging system making use of a wavelength swept light source. The OCT imaging system making use of a wavelength swept light source and the above-described OCT imaging system are basically the same in measurement principle as shown in FIGS. 14 and 15 in that they make use of optical interference. The following description thus primarily centers on differences relative to the above-described OCT imaging system.

It is a light source that is different in measurement principle from the above-described OCT imaging system. First, these OCT imaging systems are thus different in coherence length. More specifically, a light source capable of emitting low-coherence light of from 10 μm to 20 μm or so in coherence length is used in the above-described OCT imaging system, while a light source having a coherence length of from 4 mm to 10 mm or so is used in the OCT imaging system making use of wavelength swept light source.

One reason for the above-mentioned difference is that the range of the examinable range in the direction of the depth of a biotissue is dependent on the movable range of the reference mirror in the above-described OCT imaging system, but is dependent on the coherence length in the OCT imaging system making use of wavelength swept light source. To encompass the entire range in the direction of the depth of a biotissue such as a blood vessel, a light source having a relatively long coherence length is used in the OCT imaging system making use of wavelength swept light source.

A second difference in their light sources resides in that in the case of the OCT imaging system making use of wavelength swept light source, light beams having different wavelengths are continuously irradiated.

In the above-described OCT imaging system, the extraction of reflected light from individual points in the direction of the depth of the biotissue is achieved by movements of the reference mirror, and the resolution in the direction of the depth of the measurement target is dependent on the coherence length of irradiated light.

The OCT imaging system making use of wavelength swept light source, on the other hand, is characterized in that light is irradiated while continuously varying its wavelength and the intensities of reflected light from individual points in the direction of the depth of the biotissue are determined based on differences in the frequency component of interference light.

Taking the frequency (the inverse of the wavelength) of scanning light as a time function represented by Equation 1, the intensity of interference light can generally be expressed by a time function represented by Equation 2.

f(t)=f_{α}+Δft (Equation 1)

I(t)=A+B cos (CΔx(f_{α}+Δft)) (Equation 2)

where Δx: optical path difference between the reference light and the target light,
Δf: the rate of a change in frequency in unit time, and

### A,B,C: constants.

As appreciated from Equation 2, the frequency component in the time-dependent change in the intensity I(t) of reference light is expressed by the optical path difference Δx and the rate Δf of a change in frequency by wavelength sweeping. Accordingly, the intensity of interference light for each optical path difference can be determined provided that the frequency component of the interference light is known.

As a consequence, the time required for acquiring signals for one line can be shortened, and further, the imaging depth can be made greater.

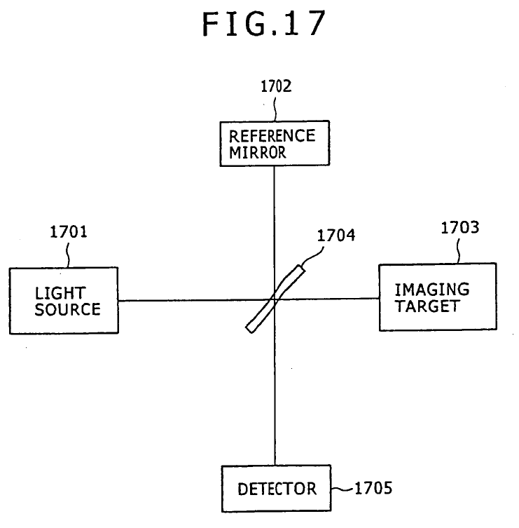

A schematic illustration of the basic principle of the above-described OCT imaging system making use of wavelength swept light source is illustrated in FIG. 17. In this illustrated embodiment, the light source 1701 is a swept laser.

Light beams, which have been successively outputted from the light source 1701 and have different wavelengths, are each split at a beam splitter 1704, and the thus-split light beams then travel toward a reference mirror 1702 (i.e., reference optical path) and an imaging target 1703 (i.e., sample optical path), respectively. At this time, reflected light which is returning from the side of the imaging target 1703 includes light reflected on the surface of the imaging target, light reflected at shallow points in the imaging target, and light reflected at deep points in the imaging target.

By subjecting observed reference light to frequency resolution at a detector 1705 as mentioned above, information on a structure at a particular position in the direction of the depth of the measuring target can be visualized. As a result, a tomographic image can be formed.

As the light outputted from the light source 1701 is of from 4 to 10 mm or so in coherence length, it is possible to encompass the entire examination range in the direction of the depth of the imaging target. It is, therefore, unnecessary to move the reference mirror, so that the reference mirror 1702 is arranged fixedly at a constant distance. Moreover the reference mirror is not indispensable in this embodiment. A turned optical fiber, which can return back the light, may be set at the distal end of the reference optical path instead of the reference mirror.

Because it is unnecessary to mechanically move the reference mirror as mentioned above, the OCT imaging system making use of wavelength swept light source, in comparison with the above-described OCT imaging system, requires a shorter time for acquiring signals for one line and can raise the frame rate. As opposed to a maximum frame rate of 15 fr/s (frames/second) in the above-described OCT imaging system, the frame rate of the OCT imaging system making use of wavelength swept light source is as high as from 30 to 200 fr/s or so.

In the case of an OCT imaging system, irrespective of whether or not it makes use of wavelength swept light source, blood is supposed to be eliminated upon diagnosis so that absorption of light by blood cell components can be avoided to acquire good images. A low frame rate, therefore, requires the elimination of blood for a longer time. This, however, can lead to problems from the clinical standpoint. In the case of an OCT imaging system making use of wavelength swept light source, images can be acquired over 30 mm or longer in the axial direction of a blood vessel by elimination of blood for several seconds, thereby reducing such clinical concerns.

### 5. Aspects and Features of the OCT Imaging System Making Use of a Wavelength Swept Light Source

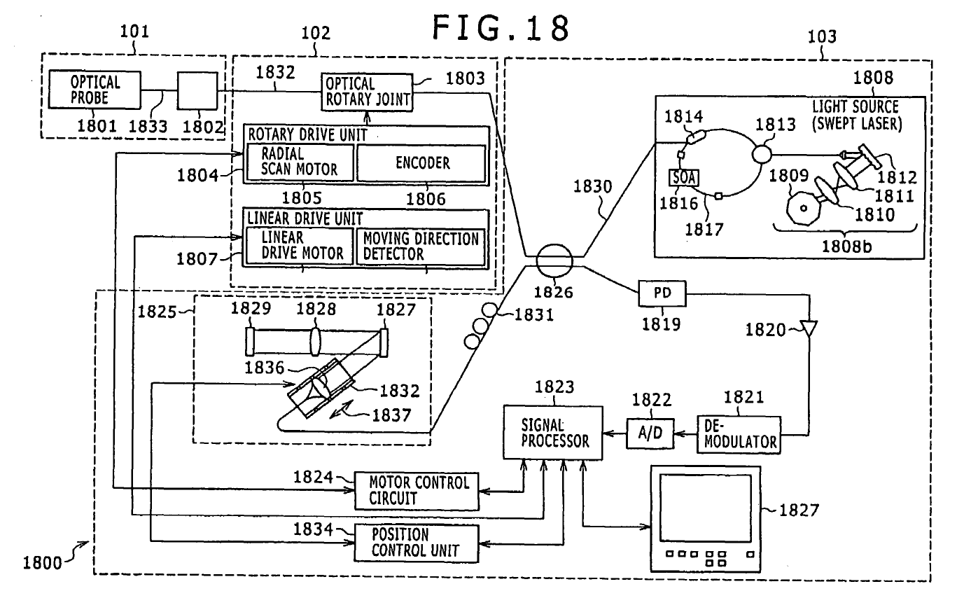

Features and aspects of the OCT imaging system 1800 according to a modification of the above-described second embodiment which makes use of a wavelength sweeping are schematically shown in FIG. 18. The description which follows primarily describes differences in the OCT imaging system 1800 making use of a wavelength swept light source relative to the OCT imaging system described above and illustrated in FIG. 16 as the second embodiment.

The OCT imaging system making use of wavelength swept light source includes a light source 1808 with a swept laser used as the optical source 1808. This swept laser 1808 is a kind of extended-cavity laser, which includes an optical fiber 1817 and a polygon scanning filter 1808b. The optical fiber 1817 is connected in the form of a ring with a semiconductor optical amplifier (SOA) 1816.

Light outputted from the SOA 1816 advances through the optical fiber 1817, and enters the polygon scanning filter 1808b. Subsequent to wavelength selection through the polygon scanning filter 1808b, the resulting light is amplified at the SOA 1816 and is finally outputted from a coupler 1814.

The polygon scanning filter 1808b selects a wavelength by a combination of a diffraction grating 1812, which separates light into a spectrum, and a polygon mirror 1809. The light which has been separated into the spectrum by the diffraction grating 1812 is condensed on a facet of the polygon mirror 1809 by two lenses (1810, 1811). As a result, only light of a wavelength crossing at a right angle with the polygon mirror 1809 returns on the same light path and is outputted from the polygon scanning filter 1808b. By rotating the mirror, time sweeping of wavelengths is performed.

As an example of the polygon mirror 1809, a 32-sided polygonal mirror can be used, and its rotational speed can be 50,000 rpm or so. By the unique wavelength-sweeping system making the combined use of the polygon mirror 1809 and the diffraction grating 1812, high-speed and high-output wavelength sweeping is feasible.

The light of the swept laser 1808, which has been outputted from the coupler 1814, impinges on a proximal end of a first single mode fiber 1830, and is transmitted toward its distal end face. At an optical coupler 1826 arranged midway along the first single mode fiber 1830, the first single mode fiber 1830 is optically coupled with a second single mode fiber 1831. Therefore, the light transmitted through the first single mode fiber 1830 is split into two by the optical coupler 1826 and the resulting two beams of light are transmitted further.

On the more distal end side of the first single mode fiber 1830 than the optical coupler 1826 (i.e., reference optical path), an optical rotary joint 1803 is arranged to connect a non-rotatable block and a rotatable block with each other such that light can be transmitted.

Further, an optical-probe connector 1802 is detachably connected to a distal end of a third single mode fiber 1832 in the optical rotary joint 1803. Via the connector 1802, the light from the light source 1808 is transmitted to a fourth single mode fiber 1833, which is inserted in an optical probe 1801 and is rotationally drivable.

The transmitted light is irradiated from a distal end side of the optical probe 1801 toward the surrounding biotissue of the body cavity while performing radial scanning. A portion of reflected light scattered on a surface or interior of the biotissue is collected by the optical probe 1801, and returns to the side of the first single mode fiber 1830 through the reverse optical path. A portion of the thus-collected, reflected light is transferred by the optical coupler 1826 to the side of the second single mode fiber 1831, and is introduced into a photodetector (for example, photodiode 1819) from an end of the second single mode fiber 1831. It is to be noted that the rotatable block side of the optical rotary joint 1803 is rotationally driven by a radial scan motor 1805 of the rotary drive unit 1804. Further, rotation angles of the radial scan motor 1805 are detected by an encoder 1806. The optical rotary joint 1803 is provided with a linear drive unit 1807, which based on an instruction from a signal processor 1823, controls a movement of the catheter section 101 in the direction of its insertion.

On the side of a more distal end of the second single mode fiber 1831 than the optical coupler 1826, an optical path length (OPL) varying mechanism 1825 is arranged to finely adjust the optical path length of reference light.

This OPL varying mechanism 1825 is provided with a an OPL varying means for varying the optical path length by a length equivalent to a variation in the length of a new optical probe to absorb the variation when the new optical probe is used as a replacement.

The second single mode fiber 1831 and a collimator lens 1836 are mounted on a single axis stage 1835 movable in the direction of an optical axis of the collimator lens 1836 as indicated by an arrow 1837, thereby forming the OPL varying mechanism.

More specifically, the single axis stage 1835 forms the OPL varying mechanism having a variable OPL range just enough to absorb a variation in the optical path length of a new optical probe when the optical probe 1801 is replaced by the new optical probe. In addition, the single axis stage 1835 is also equipped with a function as an adjustment means for adjusting an offset. Even when the distal end of the optical probe 1801 is not in close contact with a surface of the biotissue, for example, the optical probe can still be set in such a state as interfering from a position on the surface of the biotissue by slightly varying the optical path length with the single axis stage 1835.

The light finely adjusted in optical path length by the OPL varying mechanism 1825 is combined with the light, which has escaped from the side of the first single mode fiber 1830, at the optical coupler 1826 arranged midway along the second single mode fiber 1831, and the combined light is received at the photodiode 1819.

The light received at the photodiode 1819 is photoelectrically converted, amplified by an amplifier 1820, and then inputted into a demodulator 1821. At the demodulator 1821, demodulation processing is performed to extract only the signal portion of the interfered light, and the output of the demodulator 1821 is inputted into an A/D converter 1822.

At the A/D converter 1822, interference light signals are sampled at 180 MHz as much as for 2,048 points to produce digital data (interference light data) for one line. It is to be noted that the setting of the sampling frequency at 180 MHz is attributed to the premise that approximately 90% of the cycle of wavelength sweeping (12.5 μsec) be extracted as digital data at 2,048 points when the wavelength sweep repetition frequency is set at 40 kHz. The sampling frequency should, therefore, not be limited specifically to the above-described value.

The interference light data in the line unit, which have been produced at the A/D converter 1822, are inputted into a signal processor 1823. At this signal processor 1823, the interference light data are frequency-resolved by FFT (Fast Fourier Transform) to produce data in the direction of the depth. These data are then coordinate-transformed to construct tomographic images at respective positions in the blood vessel. The tomographic images are then outputted at a predetermined frame rate to an LCD monitor 1827.

It is to be noted that the signal processor 1823 is connected with a position control unit 1834. The signal processor 1823 performs control of the position of the single axis stage 1835 via the position control unit 1834. In addition, the signal processor 1823 is also connected with a motor control circuit 1824, and in synchronization with video synchronization signals upon formation of tomographic images, stores the tomographic images in its internal memory.

### 6. Features of Distal End Portion of Catheter Section

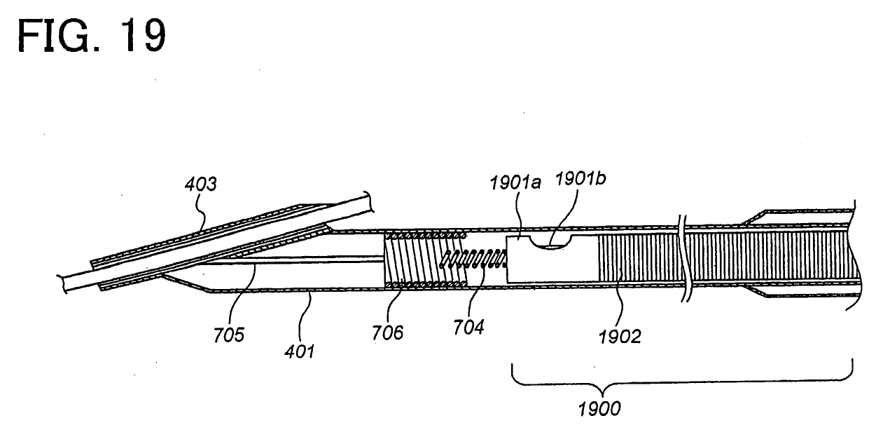

The overall construction of the catheter section 101 is the same as the construction (FIG. 4 and FIG. 5) of the catheter section in the IVUS imaging system described above in the first embodiment, and so such description is not repeated here. Referring to FIG. 19, the following description primarily describes differences in the construction of the distal end portion of the catheter section 101.

FIG. 19 is a cross-sectional view showing the construction of the distal end portion of the catheter section 101 used in the OCT imaging system 1600 according to the second embodiment and in the OCT imaging system 1800 making use of a wavelength swept light source according to the modification of the second embodiment (i.e., the third embodiment).

Referring to FIG. 19, an imaging core 1900 extends through the lumen of the catheter sheath 401 over substantially the entire length of the catheter sheath 401. The imaging core 1900 comprises a driveshaft 1902 for transmitting drive force. The driveshaft 1902 is a hollow shaft constructed in the form of a coil and accommodates an optical transmission line (e.g., optical fiber) in the hollow portion (i.e., lumen). The optical fiber (not illustrated) transmits optical signals (i.e., light signals). A prism or mirror 1901b held in a housing 1901a is attached at the distal end of the optical fiber for irradiating and receiving the light signals. The imaging core 1900 irradiates light toward a surrounding biotissue of a body cavity from the prism or mirror 1901b, and at the prism or mirror 1901b, receives reflected light from the surrounding biotissue of the body cavity by the radial scanning. The optical fiber is disposed through the driveshaft 1902, and extends from the housing 1901a to the connector 1602 or 1802.

As the advance injection of physiological saline (priming work) is not absolutely needed in the OCT imaging system according to the second embodiment or the OCT imaging system making use of wavelength swept light source according to the modification of the second embodiment, the priming discharge channel 705 formed at the boundary portion between the distal end portion of the catheter sheath 401 and the guidewire lumen 403 may be omitted.

### 7. Construction of the Driveshaft Connector 402b

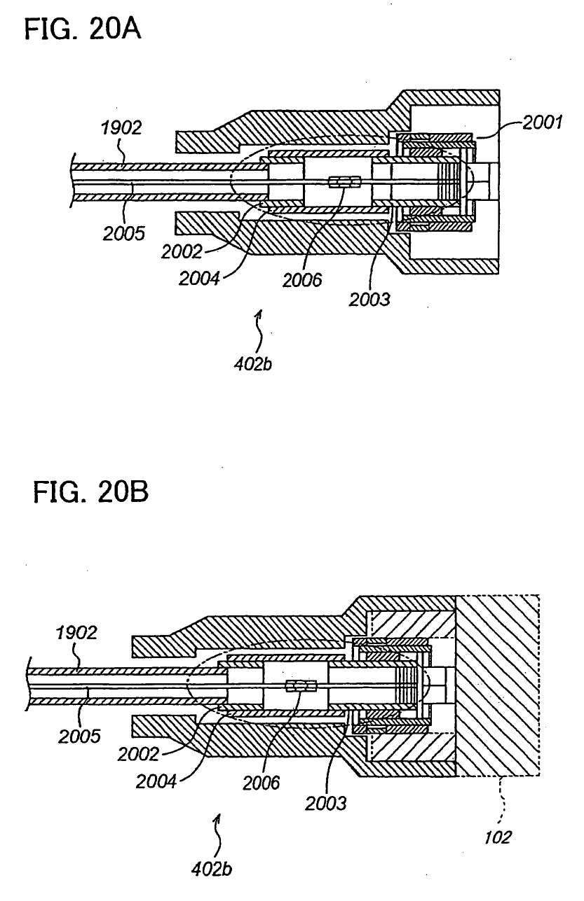

FIGS. 20A and 20B are cross-sectional views showing the internal construction of the driveshaft connector 402b. The user side (i.e., proximal side) of the driveshaft 1902 is externally covered by a sheath hub, and the sheath hub is constructed to permit easy connection with the scanner & pull-back unit 102. FIG. 20A shows the driveshaft connector 402b without the scanner & pull-back unit 102, and FIG. 20B illustrates the driveshaft connector 402b with the scanner & pull-back unit 102 connected to the driveshaft connector 402b.

As depicted in FIGS. 20A and 20B, the driveshaft 1902 and a connector 2001 are coupled with each other at a torque limiter 2004 via a distal-end-side pipe 2002 and a user-side pipe 2003. Further, an optical fiber 2005 is connected to the scanner & pull-back unit 102 via the connector 2001.

In addition, a portion of the optical fiber 2005 inside the torque limiter 2004 is formed as a fusion-spliced portion (details of which will be described below), thereby providing a mechanism by which the rotational drive force transmitted from the scanner & pull-back unit 102 is cut off by the destruction of the torque limiter 2004 when a load torque of a predetermined value or greater is applied.

The construction of the torque limiter 2004 is the same as that of the torque limiter 804 described above in the first embodiment and so a detailed description is not repeated.

### 8. Construction of Fusion-Spliced Portion 2006

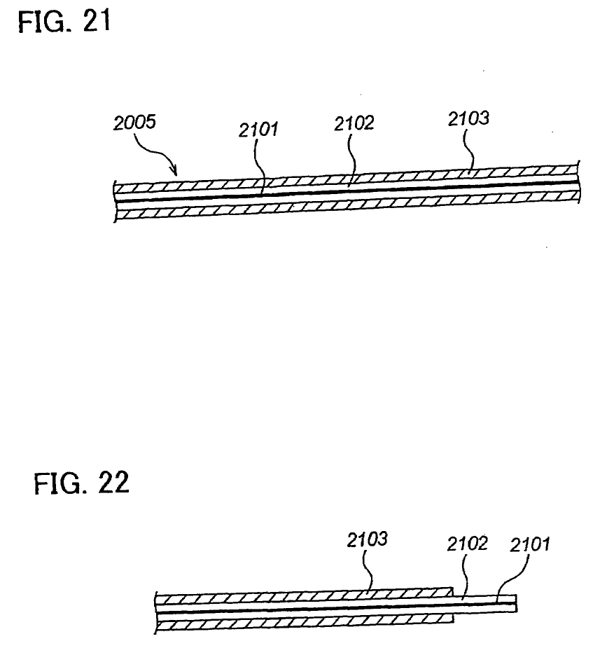

Details associated with the fusion-spliced portion 2006 of the optical fiber 2005 are illustrated in FIGS. 21-23C.

### 8.1 Construction of the Optical Fiber

FIG. 21 illustrates features, in cross-section, of the general single-mode optical fiber. The optical fiber 2005 is composed of a core 2101 for transmitting light and a cladding 2102 having a slightly smaller refractive index than the core 2101. Only when an incidence angle is greater than a critical angle, light is transmitted while repeating total reflection on an interface surface between the core 2101 and the cladding 2102. The outer surface of the cladding 2102 of the optical fiber 2005 is covered by a resin material referred to as a jacket 2103 so that, even when the optical fiber 2005 is bent with a large curvature, the resulting stress is dispersed to protect the optical fiber 2005 from breakage.

Optical fibers themselves can be connected with each other by using an optical fiber splicing machine which is widely used in the communication industry. The term “optical fiber splicing machine” means a machine for fusion-splicing optical fibers with heat produced by an arc discharge.

FIG. 22 illustrates a prepared state of the end face of the optical fiber before setting it on the optical fiber splicing machine. At an end portion of the optical fiber, the jacket 2103 is stripped off beforehand by a special-purpose tool (not shown) called a jacket stripper, and then end face of the cladding 2102 is perpendicularly cut in advance by a special-purpose tool (not shown) called a cleaver.

### 8.2 Fusion-Splicing of Optical Fibers

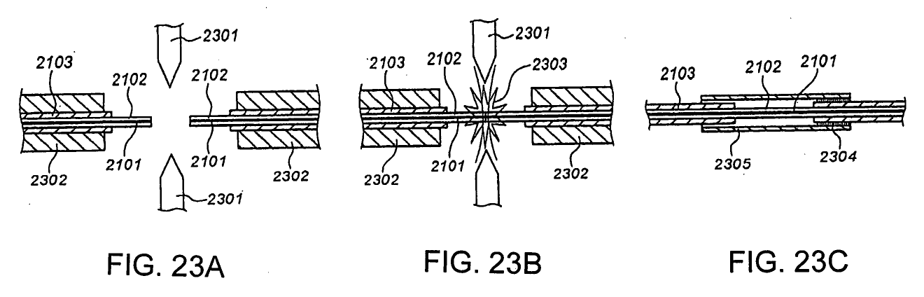

The way in which the optical fibers are fusion-spliced using the optical fiber splicing machine is generally illustrated in FIGS. 23A-23C. FIG. 23A illustrates the optical fibers set on the optical fiber splicing machine. As depicted in FIG. 23A, the optical fibers are fixed on fiber holders 2302 and are positioned between opposing electrodes 2301 of the optical fiber splicing machine. As illustrated, the optical fibers with the claddings 2102 exposed at their end portions are positioned in opposing relation to each other in a direction perpendicular to an imaginary line extending between the two electrodes 2301.

FIG. 23B illustrates an arc discharge produced between the electrodes of the optical fiber splicing machine and the optical fibers being fusion-spliced. By switching operations of the optical fiber splicing machine, the respective optical fibers are automatically brought into alignment and close to each other on an automated stage, and subsequently, an arc discharge 2303 is produced between the electrodes 2301 to achieve fusion-splicing of the optical fibers themselves.

FIG. 23C depict a capillary tubing 2305 applied subsequent to the fusion-splicing of the optical fibers. As the fusion-spliced portion is lower in durability than the other portions of the optical fibers, the fusion-spliced portion is covered and protected by the capillary tubing 2305. The material of this capillary tubing 2305 is preferably a resin material, more preferably a resin material having good adhesion property with general epoxy-based adhesives, cyanoacrylate-based adhesives and UV-curable adhesives. Still more preferably, a transparent or semitransparent resin material is used which makes it possible to see the inside through the capillary tubing 2305. As a preferred example, polyimide can be selected as the material of the capillary tubing 2305. With an adhesive 2304, the capillary tubing 2305 is fixedly united with the jacket 2103 on either the user side or the distal-end-side. In this illustrated and disclosed embodiment, the capillary tubing 2305 is fixedly united (adhered) on its user side. The fusion-spliced portion 2006 is thus formed as described above.

### 8.3 Functions of the Torque Limiter 2004 and the Fusion-Spliced Portion 2006

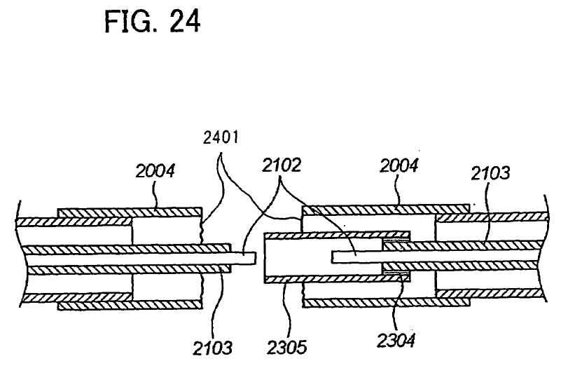

FIG. 24 illustrates a situation in which the torque limiter 2004 and optical fiber 2005 have broken off as a result of an application of an overload to the distal end portion of the driveshaft. When a load torque of a predetermined value or greater is applied to the distal end portion of the driveshaft 1902, the rotational drive force from the scanner & pull-back unit 102 to the driveshaft 1902 is cut off and at the same time the fusion-spliced portion of the optical fiber is broken off as generally indicated at numeral 2401.

In the OCT imaging system according to the second embodiment and the OCT imaging system making use of a wavelength swept light source according to the modification of the second embodiment, the torque limiter and the fusion-spliced portion of the optical fiber are broken when an overload is applied to the distal end portion of the driveshaft. It is, therefore, possible to instantaneously cut off the rotational drive force from the driveshaft and reliably stop the rotation of the optical probe.

In the first embodiment, the second embodiment and the modification of the second embodiment, the catheters for the IVUS imaging system and the OCT imaging systems are described. However, the subject matter disclosed here is not limited to such catheters. For example, the mechanisms of the driveshaft connectors described in the first embodiment, the second embodiment and the modification of the second embodiment can be applied to a driveshaft connector in a catheter with an ultrasonic transducer unit and an optical probe accommodated in combination therein. Such an embodiment can be realized by combining the torque limiter, the torque-limiting connector for the electric transmission line and the fusion-spliced portion of the optical fiber, all of which were described above.

The principles, preferred embodiments and modes of operation have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. An image diagnostic system comprising:
a probe positionable in a body cavity and configured to repeatedly transmit signals and acquire signals reflected from biotissue surrounding the body cavity during radial scanning;
a scanner and pullback unit connected to the probe to rotate and axially move the probe during the radial scanning;
a torque limiter positioned to limit a torque load applied to the probe by the scanner and pullback unit;
the torque limiter comprising a shaft portion provided with a plurality of circumferentially arranged grooves which cause the shaft portion to break when the torque load applied by the scanner and pullback unit exceeds a predetermined load;
a control unit connected to the probe by way of a transmission line to produce digital data based on the acquired signals and to construct a tomographic image of the body cavity and the biotissue surrounding the body cavity on the basis of the digital data; and
a display unit connected to the control unit to display the tomographic image.

2. The image diagnostic system according to claim 1, wherein at least some of the grooves are through-grooves which extend completely through a wall of the shaft portion.

3. The image diagnostic system according to claim 1, wherein at least some of the grooves do not extend completely through a wall of the shaft portion.

4. The image diagnostic system according to claim 1, wherein the transmission line is an electric transmission line comprised of two parts detachably connected in the torque limiter.

5. The image diagnostic system according to claim 1, wherein the transmission line is an optical fiber cable comprised of two parts fusion-spliced in the torque limiter.

6. An image diagnostic system comprising:
a probe positionable in a body cavity and configured to repeatedly transmit signals and acquire signals reflected from biotissue surrounding the body cavity during radial scanning;
a control unit connected to the probe to produce digital data based on the acquired signals and to construct a tomographic image of the body cavity and the biotissue surrounding the body cavity on the basis of the digital data; and
a display unit configured to display the tomographic image;
the probe comprising:
a shaft transmitting a rotational drive force during the radial scanning by the probe;
a transmission line extending along the shaft to transmit the reflected signals to the control unit;
the shaft receiving the rotational drive force via a torque limiter; and
the torque limiter possessing a thickness which is non-uniform in a circumferential direction of the torque limiter at a part along a length of the torque limiter.

7. The image diagnostic system according to claim 6, wherein the torque limiter comprises a cylindrical body with intermittent through-slots formed in a circumferential direction of the cylindrical body at a part along the length of the cylindrical body.

8. The image diagnostic system according to claim 6, wherein the torque limiter comprises a cylindrical body with intermittent non-through-slots formed in a circumferential direction of the cylindrical body at a part along the length of the cylindrical body.

9. The image diagnostic system according to claim 6, wherein the torque limiter comprises a cylindrical body with a continuous non-through-slot formed in a circumferential direction of the cylindrical body at a part along the length of the cylindrical body.

10. The image diagnostic system according to claim 6, wherein the transmission line is an electric transmission line.

11. The image diagnostic system according to claim 10, wherein the electric transmission line comprises two parts detachably connected in the torque limiter.

12. The image diagnostic system according to claim 6, wherein the transmission line is an optical fiber cable.

13. The image diagnostic system according to claim 12, wherein the optical fiber cable comprises two parts fusion-spliced in the torque limiter.

14. A probe connectable to an image diagnostic apparatus and positionable in a body cavity comprising:
an imaging core for transmitting signals and receiving reflected signals used by the image diagnostic apparatus to produce digital data for constructing a tomographic image of the body cavity and biotissue surrounding the body cavity;
the image core comprising a shaft configured to transmit rotational drive force to a distal portion of the imaging core;
a transmission line extending along the shaft to transmit the reflected signals to the control unit;
a torque limiter positioned at a portion of the shaft to limit a torque load transmitted to the distal portion of the imaging core; and
the torque limiter possessing a thickness which is non-uniform in a circumferential direction of the torque limiter at a part along a length of the torque limiter.

15. The probe according to claim 14, wherein the torque limiter comprises a cylindrical body with intermittent through-slots formed in a circumferential direction of the cylindrical body at a part along the length of the cylindrical body.

16. The probe according to claim 14, wherein the torque limiter comprises a cylindrical body with intermittent non-through-slots formed in a circumferential direction of the cylindrical body at a part along the length of the cylindrical body.

17. The probe according to claim 14, wherein the torque limiter comprises a cylindrical body with a continuous non-through-slot formed in a circumferential direction of the cylindrical body at a part along the length of the cylindrical body.

18. The probe according to claim 14, wherein the transmission line is an electric transmission line.

19. The probe according to claim 18, wherein the electric transmission line comprises two parts detachably connected in the torque limiter.

20. The probe according to claim 14, wherein the transmission line is an optical fiber cable.

21. The probe according to claim 20, wherein the optical fiber cable comprises two parts fusion-spliced in the torque limiter.

22. A catheter comprising:
a sheath possessing a lumen;
a shaft positioned in the lumen and configured to transmit a rotational drive force to a distal portion; and
a torque limiter positioned at a proximal portion of the shaft to transmit the rotational drive force when the rotational drive force is less than a predetermined value, the torque limiter possessing a vulnerable portion along a circumferential direction that breaks upon application of a load torque equal to or greater than the predetermined value to prevent transmission of the torque load equal to or greater than the predetermined value to the distal portion.

23. The catheter according to claim 22, wherein the torque limiter comprises a cylindrical tube and the vulnerable portion comprises a circumferentially arranged portion of the cylindrical tube possessing a non-uniform thickness.
